(19) United States
(12) Patent Application Publication
(10) Pub. No.: US 2026/0355125 A1
(43) Pub. Date: Nov. 12, 2026

(54) ELECTROSPUN MULTILAYERED NANOFIBER-NANOPARTICLE MEMBRANE DRUG CARRIER

(71) Applicant: UNIVERSITY OF SOUTH FLORIDA, Tampa, FL (US)

(72) Inventors: Ridita KHAN, Tampa, FL (US); Sylvia THOMAS, Tampa, FL (US)

(21) Appl. No.: 19/643,542

(22) Filed: Apr. 9, 2026

Related U.S. Application Data
(60) Provisional application No. 63/786,039, filed on Apr. 9, 2025.

(51) Int. Cl. A61K 9/70; A61K 9/00; A61K 31/704; A61K 47/52; A61K 47/59

(52) U.S. Cl. CPC A61K 9/7007; A61K 9/0002; A61K 31/704; A61K 47/52; A61K 47/593

(57) ABSTRACT

Described herein are electrospun biodegradable flexible nanofiber membranes that retain therapeutic-loaded nanoparticles within fibers, providing localized drug delivery suitable for post-operative treatment of solid tumors. Poly(lactic-co-glycolic acid) fibers retain doxorubicin-loaded mesoporous silicon dioxide nanoparticles to form a drug-in-nanoparticle-in-fiber construct that provides a dual-barrier release architecture governed by nanoparticle pore diffusion and polymer-matrix diffusion and degradation to suppress burst release and provide sustained delivery. The flexible nanofiber membrane comprises coaxial or triaxial fibers that compartmentalize different payloads to provide staged time-release profiles, including a delayed-start release mediated by acidic pH lower than physiological pH, using a pH-responsive layer that increases porosity or permeability under acidic conditions. Representative methods include surfactant-templated synthesis of mesoporous silica nanoparticles, drug loading, dispersion into polymer solutions, electrospinning to form flexible nanofiber membranes, and in vitro characterization of morphology, chemical composition, release behavior, and cellular uptake in a non-small cell lung cancer cell model.

### CROSS REFERENCE TO RELATED APPLICATIONS

This application claims the benefit of and priority to U.S. Provisional Application No. 63/786,039, filed Apr. 9, 2025, titled “ELECTROSPUN MULTILAYERED NANOFIBER-NANOPARTICLE MEMBRANE DRUG CARRIER” the entire disclosure of which is incorporated by reference.

### FIELD

This disclosure relates to biomedical engineering, implantable drug delivery, electrospun fiber constructs, and localized chemotherapy for treatment of solid tumors including non-small cell lung cancer.

### BACKGROUND

Non-small cell lung cancer remains a leading contributor to cancer-related mortality and frequently presents recurrence risk following surgical resection. Systemic chemotherapy exposes non-target tissues to cytotoxic agents and contributes to dose-limiting toxicity. Localized delivery at a resection margin supports high local drug exposure while reducing systemic exposure.

Electrospinning provides a scalable route to produce nano- to micro-scale fibers with high surface area, tunable porosity, and controllable architecture. Multi-axial electrospinning supports core-shell and multi-layer fibers that compartmentalize payloads and shape release kinetics. Despite progress, a need remains for implantable, biodegradable fiber platforms that (i) retain therapeutic-loaded nanoparticles within an electrospun fiber matrix to limit nanoparticle migration and systemic distribution, and (ii) provide controlled release using a dual-barrier architecture that combines nanoparticle pore diffusion with polymer-matrix diffusion and degradation, thereby reducing burst release and enabling programmable local therapy.

### SUMMARY

This disclosure describes an implantable, biodegradable nanofiber membrane that includes electrospun polymer fibers and therapeutic-loaded nanoparticles distributed within the fibers. In representative implementations, a poly(lactic-co-glycolic acid) (PLGA) fiber matrix contains doxorubicin-loaded mesoporous silicon dioxide nanoparticles (DOX@SiO₂), providing a dual-barrier release architecture that suppresses burst release and supports sustained local delivery. Doxorubicin (DOX), is known as a potent chemotherapeutic, used to treat various cancers.

As described herein, the membrane provides a drug-in-nanoparticle-in-fiber construct in which a small-molecule therapeutic is retained within pores of mesoporous silicon dioxide nanoparticles and the nanoparticles are retained within biodegradable electrospun fibers, such that release is governed by both nanoparticle desorption/diffusion and polymer-matrix diffusion and degradation. In one embodiment, the membrane further provides staged release by compartmentalizing different payloads in different regions of coaxial or triaxial fibers, and a delayed-start release is triggered by a stimulus-responsive layer that surrounds a compartment containing a payload to be released under tumor-microenvironment conditions.

The membrane supports multi-axial fiber architectures including coaxial and triaxial fibers that compartmentalize different therapeutic agents, functional molecules, or gene delivery components in discrete layers. The membrane is configured for placement at a tumor bed or surgical margin, where drug release provides local exposure to residual malignant cells while reducing systemic distribution relative to systemic dosing.

Methods described herein include nanoparticle synthesis and drug loading, electrospinning of composite solutions to form nanofiber membranes, in vitro characterization of fiber morphology and chemical composition, and in vitro assays that quantify drug release and cellular uptake.

### BRIEF DESCRIPTION OF THE FIGURES

FIG. 1 depicts a model of triaxial nanofiber. (A) Exterior view of a triaxial nanofiber. (B) Cross section of a triaxial nanofiber designed to carry a multiple drug payload and therapeutics.

FIG. 2 depicts a schematic of electrospinning used for nanofiber fabrication.

FIG. 3 depicts a top view of an electrospinning setup.

FIG. 4 depicts a top view of multiaxial electrospinning setup.

FIG. 5 depicts SEM image of electrospun nanofibers. (A) PLGA nanofibers. (B) PEO nanofibers. (C) PEO and PEI nanofibers (D) Tri-axil nanofibers.

FIG. 6 depicts TEM images of nanofibers. (A) Triaxial nanofiber. (B) Coaxial nanofiber.

FIG. 7 depicts a TEM image of a silicon dioxide nanoparticle at different magnification levels.

FIG. 8 depicts drug release profile of 1w % PLGA.

FIG. 9 depicts drug release profile of 5w % PLGA.

FIG. 10 shows a comparison of drug release profile of 1w % and 5w % PLGA.

FIG. 11 shows the FTIR spectrum of PLGA Membrane.

FIG. 12 shows the FTIR spectrum of DOX loaded PLGA nanofiber membrane.

FIG. 13 shows a well plate treated with 5w % polymer membrane with DOX uptake. (A) Top view of A549 treated with drug loaded membrane. (B) Bottom view of A549 treated with drug loaded membrane. (C) View of A549 cells without florescence treated with drug loaded membrane.

FIG. 14 shows a well plate treated with 1w % polymer membrane with DOX uptake. (A) Top view of A549 cells treated with drug loaded membrane. (B) Bottom view of A549 cells treated with drug loaded membrane. (C) View of A549 cells without florescence treated with drug loaded membrane.

FIG. 15 shows A549 cells treated with a polymer membrane with no drug.

FIG. 16 shows a well plate with A549 cells without polymer membrane. (A) Top view of A549 treated with no membrane. (B) Bottom view of A549 cells treated with no membrane. (C) View of A549 cells with florescence.

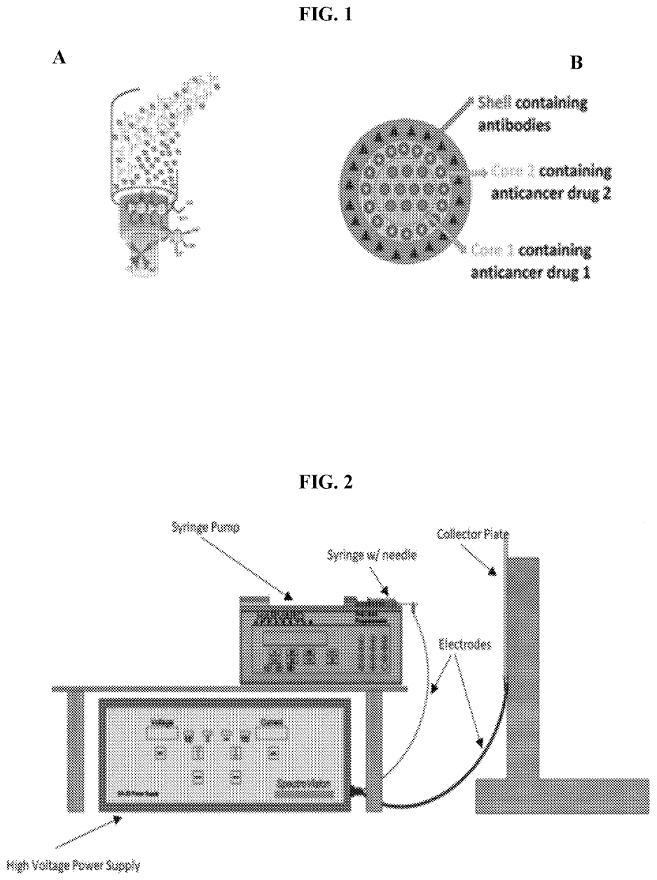

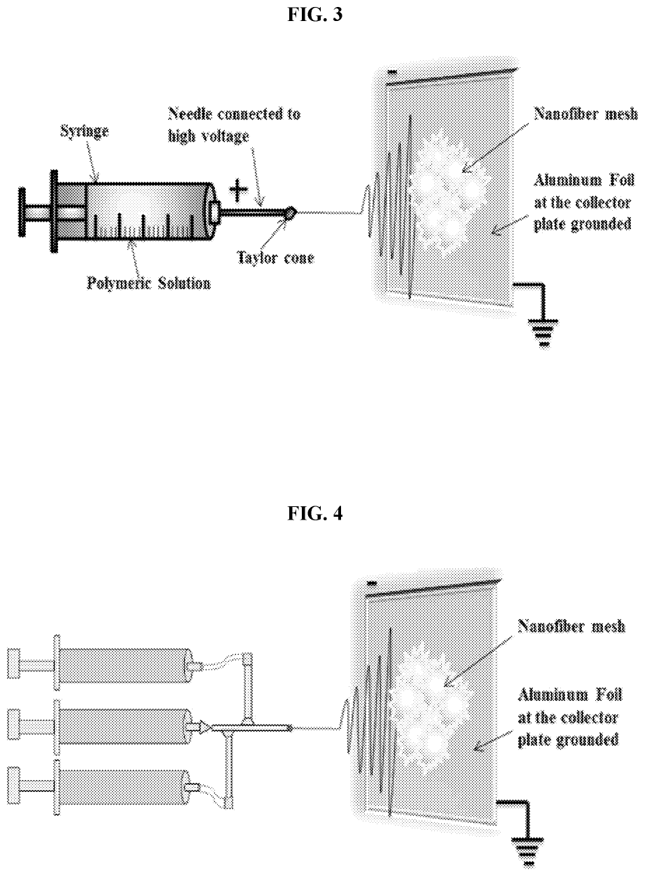

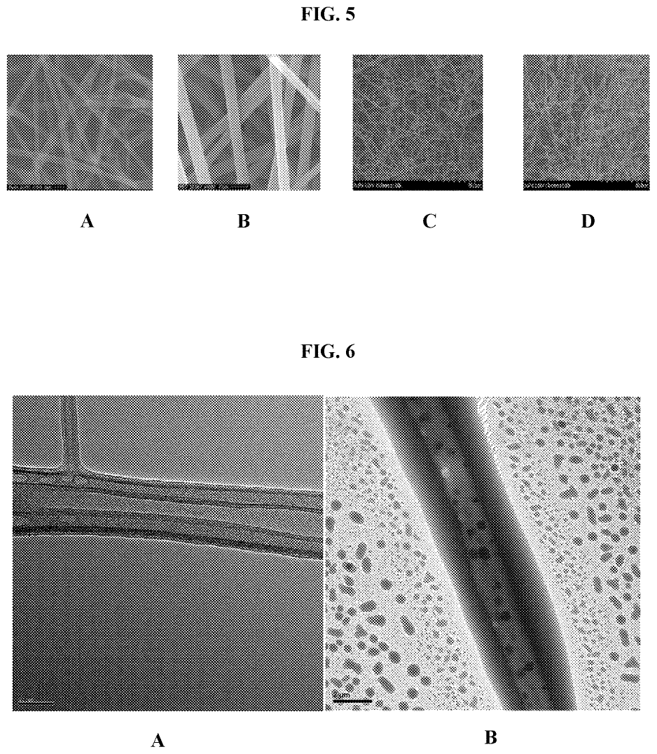

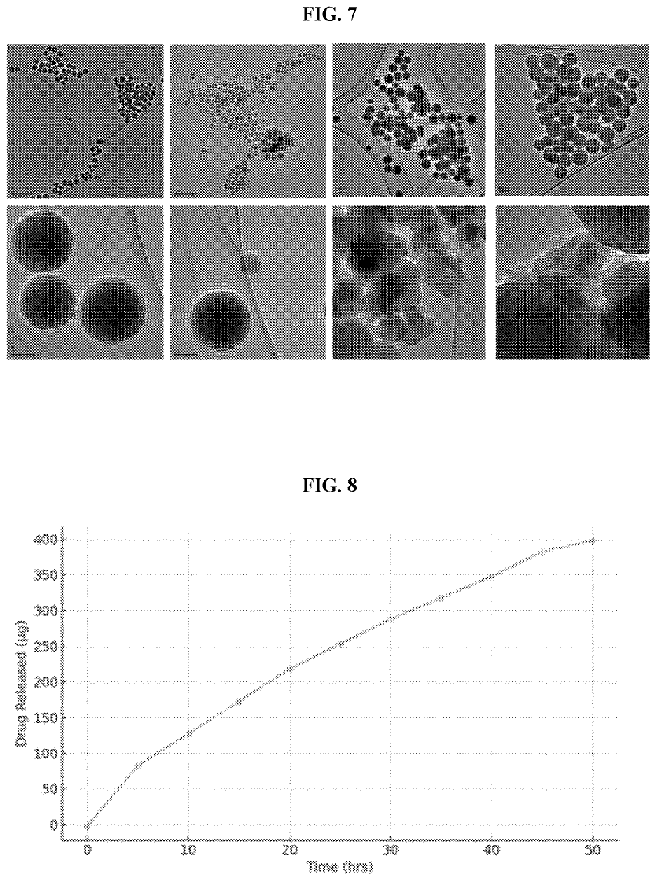

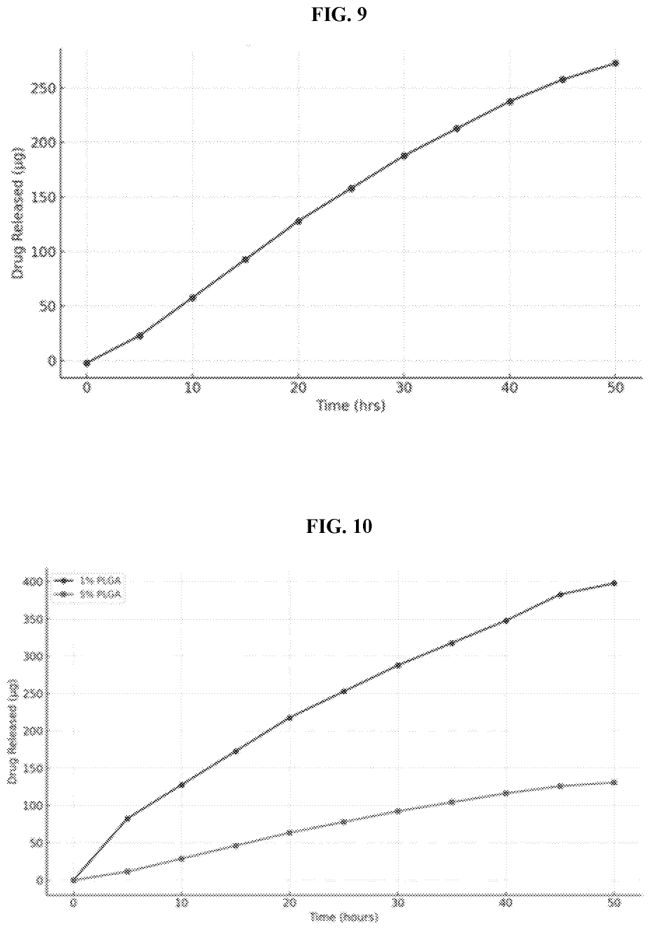

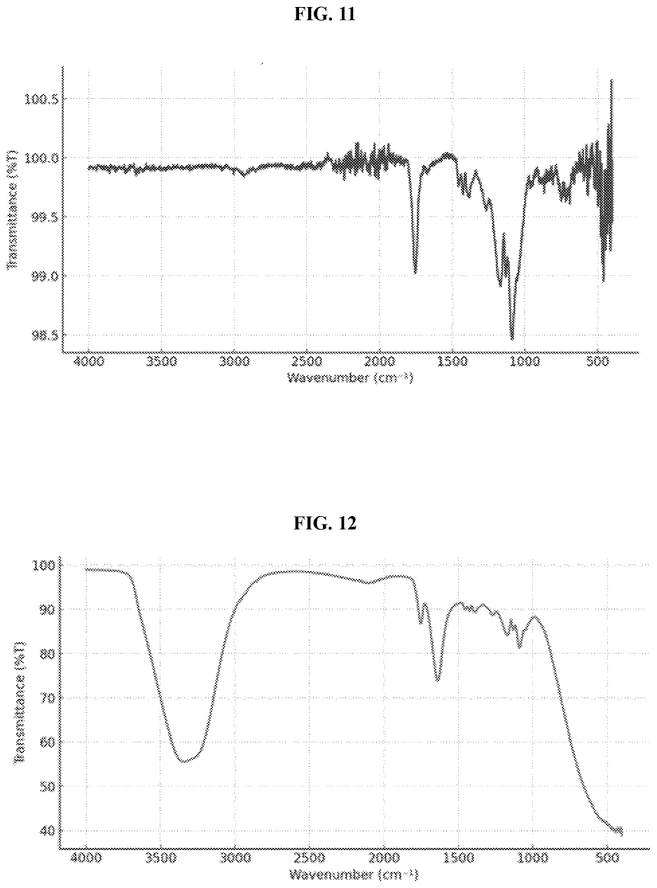

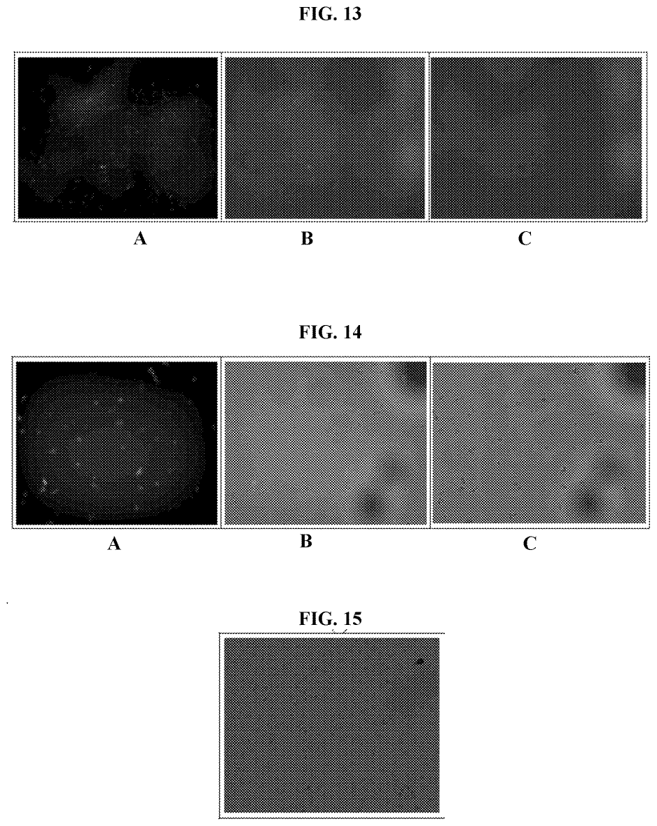

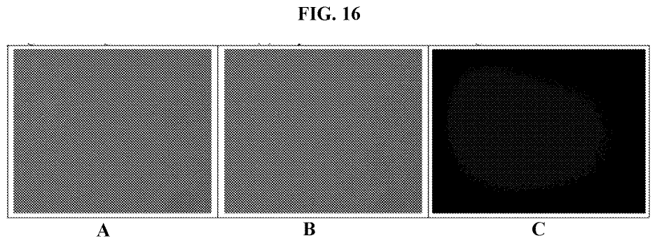

### DEFINITIONS

As used herein, the term “nanofiber membrane” refers to a sheet- or film-like construct comprising an electrospun nanofiber matrix that defines a porous fibrous network.

As used herein, the term “electrospun nanofiber matrix” refers to a fibrous matrix comprising polymer fibers formed by electrospinning a polymer-containing solution using an applied electric field.

As used herein, the term “flexible” refers to a property of a membrane that allows the membrane to conform to a tissue surface during implantation without fracturing under normal handling conditions.

As used herein, the term “biodegradable” refers to a material that degrades in vivo through chemical or enzymatic processes into byproducts that are cleared from the body over time.

As used herein, the term “therapeutic-loaded nanoparticles” refers to nanoparticles that retain or encapsulate a therapeutic payload and are retained within fibers of the electrospun nanofiber matrix.

As used herein, the term “mesoporous silicon dioxide nanoparticles” refers to silicon dioxide nanoparticles comprising internal pores that retain a therapeutic, where release occurs at least in part by desorption and diffusion from the pores.

As used herein, the term “drug-in-nanoparticle-in-fiber architecture” refers to a construct in which a drug is retained within a nanoparticle and the nanoparticle is retained within a fiber of the electrospun nanofiber matrix, such that release is governed by at least two diffusion barriers.

As used herein, the term “dual-barrier release architecture” refers to a release architecture in which release is governed by diffusion of a payload from nanoparticle pores and by diffusion through, and/or degradation of, a polymer matrix of the electrospun nanofiber matrix.

As used herein, the term “multiaxial fiber” refers to a fiber formed by coaxial electrospinning or triaxial electrospinning and comprising two or more regions arranged radially, including a core region and an outer region, and an intermediate region.

As used herein, the term “compartment” refers to a discrete region of a multiaxial fiber that retains a payload and is separated from another region by a layer that defines a diffusion resistance or degradation rate.

As used herein, the term “payload” refers to a therapeutic agent, functional molecule, or nucleic acid retained in a fiber region, nanoparticle, or layer of the nanofiber membrane.

As used herein, the term “time-release profile” refers to a release profile in which a payload is released over time and includes an initial release phase and a sustained release phase.

As used herein, the term “initial release phase” refers to an early portion of a time-release profile that establishes a therapeutic concentration at a target tissue site.

As used herein, the term “sustained release phase” refers to a later portion of a time-release profile that maintains the therapeutic concentration at the target tissue site over a longer duration.

As used herein, the term “stimulus-responsive component” refers to a component that changes permeability, porosity, or degradation rate in response to an environmental stimulus at a target tissue site.

As used herein, the term “pH-responsive component” refers to a stimulus-responsive component that changes permeability, porosity, or degradation rate in response to pH.

As used herein, the term “delayed start point” refers to a time at which release of a payload begins after placement of the nanofiber membrane at a target tissue site, where the delayed start point is later than a start of release of another payload.

As used herein, the term “targeting moiety” refers to a molecule that binds a receptor expressed by malignant cells, including an antibody, antibody fragment, peptide, aptamer, or small molecule ligand.

As used herein, the term “w %” refers to weight/volume percent (w/v) in the context of solution preparation, meaning grams of solute per 100 mL of solution.

As used herein, the term “about” when used with a numeric value refers to a variation that is within measurement and experimental error for the relevant property and measurement method.

As used herein, ‘A549’ refers to the A549 human non-small cell lung cancer cell line.

As used herein, the term “DOX@SiO₂” refers to doxorubicin loaded into mesoporous silicon dioxide nanoparticles such that doxorubicin is retained within internal pores of the mesoporous silicon dioxide nanoparticles.

### DETAILED DESCRIPTION OF THE EMBODIMENTS

In one embodiment, a drug delivery construct includes a flexible nanofiber membrane comprising an electrospun nanofiber matrix that retains therapeutic-loaded nanoparticles within fibers to provide localized therapeutic release at a target site.

In one embodiment, the electrospun nanofiber matrix comprises a biodegradable polymer selected from poly(lactic-co-glycolic acid) (PLGA), poly(L-lactic acid) (PLLA), poly(ε-caprolactone) (PCL), poly(vinyl alcohol) (PVA), poly(ethylene oxide) (PEO), poly(ethyleneimine) (PEI), poly(N-isopropylacrylamide) (pNIPAM), poly(ethylene glycol)-poly(L-lactic acid) (PEG-PLLA), or combinations thereof. (Table 1)

In one embodiment, the nanofiber matrix includes multiaxial fibers formed by coaxial or triaxial electrospinning, where a core region retains a first payload and an outer region retains a second payload to provide staged release kinetics.

In one embodiment, the multiaxial fibers include a core, an intermediate layer, and an outer layer, where each layer includes a distinct polymer composition that defines a corresponding degradation rate and diffusion resistance.

In one embodiment, the therapeutic-loaded nanoparticles comprise mesoporous silicon dioxide nanoparticles that define internal pores that retain a small-molecule therapeutic and provide controlled desorption and diffusion.

In one embodiment, the small-molecule therapeutic comprises doxorubicin or a pharmaceutically acceptable salt thereof.

In one embodiment, the nanoparticles comprise doxorubicin-loaded mesoporous silicon dioxide nanoparticles (DOX@SiO₂) dispersed within a PLGA fiber matrix to form a drug-in-nanoparticle-in-fiber architecture.

In one embodiment, the membrane includes a nanoparticle loading selected to provide a mass fraction of nanoparticles relative to polymer from 0.1% to 30% (w/w).

In one embodiment, the membrane includes a drug loading selected to provide a therapeutic dose suitable for post-operative placement at a lung resection margin.

In one embodiment, the membrane exhibits a porous morphology with fiber diameters in a range from 50 nm to 10 μm and an areal density selected to support handling and placement in a surgical field.

In one embodiment, the membrane includes an adhesive region, a suture interface, or both to support retention at a target tissue site.

In one embodiment, the membrane includes a surface functionalization that promotes tissue apposition and limits migration after placement.

In one embodiment, the membrane includes a targeting moiety selected from antibodies, antibody fragments, peptides, aptamers, or small molecules that bind receptors expressed by malignant cells.

In one embodiment, the membrane includes a stimulus-responsive component that changes permeability or degradation rate in response to pH values associated with a tumor microenvironment.

In one embodiment, the stimulus-responsive component includes a pH-responsive polymer domain, a buffering domain, or both that increase porosity under acidic conditions.

In one embodiment, the membrane provides an initial release phase that establishes therapeutic concentration at the target site followed by a sustained release phase that maintains exposure over multiple days.

In one embodiment, the sustained release phase is governed by diffusion of drug from nanoparticle pores and by hydrolytic degradation of the polymer matrix.

In one embodiment, the membrane is placed at a surgical resection bed for non-small cell lung cancer to provide local chemotherapy to residual malignant cells.

In one embodiment, the membrane is placed at a tumor bed for a solid tumor selected from liver tumors, glioma, colorectal tumors, triple negative breast tumors, cervical tumors, ovarian tumors, or combinations thereof.

In one embodiment, the membrane includes multiple therapeutics retained in different fiber layers to provide combination chemotherapy.

In one embodiment, the membrane includes a nucleic acid payload retained in a polymer layer that includes poly(ethyleneimine) to support non-viral gene delivery.

In one embodiment, the nucleic acid payload includes siRNA configured to suppress expression of a gene associated with tumor progression or drug resistance.

In one embodiment, the membrane includes a functional molecule selected from imaging probes, radiosensitizers, immune modulators, or combinations thereof to support multi-modal therapy.

In one embodiment, the membrane includes a plurality of discrete zones with different compositions to provide spatially patterned release across the membrane.

In one embodiment, the membrane degrades in vivo to biocompatible byproducts and leaves no permanent implant.

In one embodiment, a kit includes the membrane in a sterile package with instructions for placement at a tumor site and for post-placement monitoring.

In one embodiment, a method of local therapy includes placing the membrane at a target tissue site and maintaining the membrane in contact with the tissue for a duration sufficient to deliver a therapeutic dose.

In one embodiment, the method includes surgical resection of a tumor followed by placement of the membrane at a resection margin.

In one embodiment, the membrane provides multiple targeted therapies by presenting a targeting moiety at an exterior surface of the membrane and by releasing at least two therapeutic payloads from different regions of multiaxial fibers.

In one embodiment, a shell layer of a triaxial or coaxial fiber includes one or more targeting moieties selected from antibodies, antibody fragments, peptides, aptamers, or small molecules that bind to receptors expressed by malignant cells, and one or more inner layers include a first therapeutic and a second therapeutic retained in separate compartments.

In one embodiment, the first therapeutic is released according to a first time-release profile and the second therapeutic is released according to a second time-release profile that begins at a delayed start point relative to the first time-release profile, thereby providing staged delivery.

In one embodiment, the delayed start point is mediated by pH in a tumor microenvironment.

n one embodiment, a pH-responsive component is disposed in a layer that surrounds a compartment containing the second therapeutic, where the pH-responsive component increases porosity, permeability, or degradation rate when exposed to an acidic pH that is lower than physiological pH, thereby initiating release of the second therapeutic when the membrane is positioned at a tumor bed.

In one embodiment, the pH-responsive component maintains a low permeability at a pH of about 7.4 and increases permeability at a pH of about 6.8 or lower, thereby triggering release of the second therapeutic after implantation at the tumor site.

In one embodiment, these polymers are used as a shell, core, or intermediate layer of a multiaxial fiber to tune degradation rate, hydrophilicity, mechanical handling, and release kinetics.

In one embodiment, the membrane is placed at a tumor bed for a solid tumor in a tissue selected from lung tissue, liver tissue, brain tissue, colorectal tissue, breast tissue, cervical tissue, ovarian tissue, gastric tissue, or combinations thereof. In one embodiment, the tumor comprises cells selected from A549 cells, HepG-2 cells (human liver cancer), Glioma C6 cells, colorectal cancer cells, triple negative breast cancer (TNBC) cells, HeLa cells (human cervical cancer), SKOV3 cells (ovary cancer), or combinations thereof. These tissue types, tumor types, and cell-line examples are provided as non-limiting illustrations of clinical and experimental contexts in which the membranes are be used, and are not intended to limit the scope of the claims.

In one embodiment, the membrane is used to deliver a therapeutic to gastric lesions including stomach ulcers, where localized delivery provides sustained exposure at the lesion site.

In one embodiment, the flexible nanofiber membrane provides a release profile that includes an initial release phase that establishes a therapeutic concentration at a target tissue site followed by a sustained release phase that maintains the therapeutic concentration for a duration of at least 24 hours.

In one embodiment, the sustained release phase maintains therapeutic exposure over multiple days by combining diffusion of a small-molecule therapeutic from pores of mesoporous silicon dioxide nanoparticles with diffusion through, or degradation of, a biodegradable polymer matrix of the electrospun nanofiber matrix.

In one embodiment, the flexible nanofiber membrane suppresses burst release by retaining therapeutic-loaded nanoparticles within fibers of the electrospun nanofiber matrix, such that an aqueous medium contacts the polymer matrix before contacting drug retained within nanoparticle pores.

In one embodiment, the electrospun nanofiber matrix includes multiaxial fibers formed by coaxial electrospinning or triaxial electrospinning, where a core region retains a first payload and an outer region retains a second payload to provide staged release kinetics that include a first time-release profile and a second time-release profile.

In one embodiment, the second time-release profile begins at a delayed start point relative to the first time-release profile, and the delayed start point is mediated by pH values associated with a tumor microenvironment.

In one embodiment, the flexible nanofiber membrane includes a stimulus-responsive component that changes permeability or degradation rate in response to pH values associated with a tumor microenvironment, and the stimulus-responsive component increases release rate of the small-molecule therapeutic under acidic conditions relative to a release rate at physiological pH.

In one embodiment, the stimulus-responsive component includes a pH-responsive polymer domain, a buffering domain, or both that increase porosity under acidic conditions to initiate or accelerate release of a payload retained within the electrospun nanofiber matrix.

In one embodiment, a pH-responsive component is disposed in a layer that surrounds a compartment containing a second payload, where the layer increases porosity, permeability, or degradation rate when exposed to an acidic pH that is lower than physiological pH to initiate release of the second payload at the delayed start point.

In one embodiment, the pH-responsive component maintains a low permeability at a pH of about 7.4 and increases permeability at a pH of about 6.8 or lower to trigger release after implantation at a tumor site.

In one embodiment, the flexible nanofiber membrane includes a plurality of discrete zones with different compositions that provide spatially patterned release across the a flexible nanofiber membrane, and each discrete zone provides a corresponding time-release profile.

In one embodiment, the flexible nanofiber membrane includes multiple therapeutics retained in different fiber layers, and the different fiber layers define different diffusion resistances or degradation rates to provide sequential release of the multiple therapeutics.

In one embodiment, the flexible nanofiber membrane is placed at a tumor bed or surgical resection margin and provides a first local dose during the initial release phase and a maintenance dose during the sustained release phase while remaining localized to the target tissue site.

### Methods

Mesoporous silicon dioxide nanoparticles were synthesized using a surfactant-templated sol-gel process. A basic ethanol solution containing ammonium hydroxide was heated and stirred, cetyltrimethylammonium bromide (CTAB) was dissolved to form micelles, and tetraethyl orthosilicate (TEOS) was added under controlled conditions to promote hydrolysis and condensation to form silica around the micelles. The particles were isolated and treated under acidic conditions to remove CTAB and yield mesoporous particles.

Drug-loaded nanoparticles were prepared by loading doxorubicin into mesoporous silica to form DOX@SiO₂. Composite electrospinning solutions were prepared by dissolving PLGA in a solvent system comprising dichloromethane and N,N-dimethylformamide at an 80:20 volume ratio, adding DOX@SiO₂ to the polymer solution, and dispersing the nanoparticles by sonication and mixing.

Electrospinning was performed using a syringe pump, a metal needle or multiaxial spinneret, a high-voltage source, and a grounded collector. Representative parameters included a flow rate of 0.5 mL/hour, an applied voltage of approximately 13 kV, and a needle-to-collector distance of approximately 15 cm. Fibers were collected as a membrane on a collector surface and dried to remove residual solvent.

Fiber morphology and particle distribution were assessed using scanning electron microscopy and transmission electron microscopy. Chemical composition was assessed using Fourier transform infrared spectroscopy, and drug quantification was performed using ultraviolet-visible spectroscopy at 480 nm with a calibration curve that related absorbance to doxorubicin concentration.

In vitro cell studies used a human non-small cell lung cancer cell line (A549) cultured under standard conditions. Cells were exposed to drug-loaded membranes, and drug uptake was monitored using fluorescence-based imaging or related readouts.

TABLE 1
Material and Properties

| Material | Type | Properties |
|---|---|---|
| PLGA | Polymer | Biocompatible and biodegradable. FDA approved |
| | | for using in cancer drug delivery. |
| PEO | Polymer | Biocompatible and can be used for drug |
| | | delivery system. FDA Approved. |
| PEI | Polymer | Biocompatible and can be used for cancer |
| | | drug delivery. FDA Approved. |

TABLE 2
PLGA 1 w % solution preparation with 16 ml DCM and 4 ml DMF

| Concentration | PLGA | DCM | DMF | Total |
|---|---|---|---|---|
| (w %) | (g) | (mL) | (mL) | Volume (mL) |
| 1 | 0.2 | 16 | 4 | 20 |

TABLE 3
PLGA 5 w % solution preparation with 16 ml DCM and 4 ml DMF

| Concentration | PLGA | DCM | DMF | Total |
|---|---|---|---|---|
| (w %) | (g) | (mL) | (mL) | Volume (mL) |
| 5 | 1.0 | 16 | 4 | 20 |

TABLE 4
PLGA 5 w % solution preparation with 16 ml DCM and 4 ml DMF

| | 1 w % PLGA | 5 w % PLGA |
|---|---|---|
| Component | solution | solution |
| PLGA (g) | 0.2 | 1.0 |
| DCM (mL) | 16 | 16 |
| DMF (mL) | 4 | 4 |
| DOX@SiO₂ (μg/mL) | 5 | 5 |
| Solution Viscosity | Low | High |
| Expected Fiber | Uniform, smaller- | Thicker fibers, |
| Morphology | diameter fibers | more controlled |
| | | drug release |

TABLE 5
Electrospinning parameters

| Parameter | PLGA 1 w % | 5 w % PLGA solution |
|---|---|---|
| Polymer Concentration (w/v) | 1 | 5 |
| Voltage | 13 kV | 13 kV |
| Tip-to-Collector Distance | 10 cm | 18 cm |
| Expected Viscosity | Low | High |
| Expected Fiber | Thin, possibly | Thick, uniform |
| Morphology | beaded fibers | fibers |
| Solvent System | DCM:DMF | DCM:DMF |
| | (80:20 v/v) | (80:20 v/v) |

TABLE 6
Encapsulation efficiency and total drug encapsulated
in different polymer compositions

| PLGA Fiber | Initial drug | Encapsulation | Total Encapsulated |
|---|---|---|---|
| Type | added (μg) | efficiency (EE %) | Drug (μg) |
| 1 wt % PLGA | 20 | 99.60 | 19.92 |
| 5 wt % PLGA | 40 | 99.85 | 39.94 |

TABLE 7
UV-Vis drug release data for 1% PLGA + 1 μg/mL
DOX@SiO₂ nanofiber membrane in PBS (37 °C)

| Time | Absorbance | Concentration | Normalized cumulative |
|---|---|---|---|
| (hrs) | (1 w % PLGA) | (μg/mL) | drug release (%) |
| 0 | 0.000 | 0.0000 | 0.000 |
| 5 | 0.085 | 166 | 83 |
| 10 | 0.130 | 256 | 128 |
| 15 | 0.175 | 346 | 173 |
| 20 | 0.220 | 436 | 218 |
| 25 | 0.255 | 506 | 253 |
| 30 | 0.290 | 576 | 288 |
| 35 | 0.320 | 636 | 318 |
| 40 | 0.350 | 696 | 348 |
| 45 | 0.385 | 766 | 383 |
| 50 | 0.400 | 796 | 397 |

TABLE 8
UV-Vis drug release data for 5% PLGA + 5 μg/mL
DOX@SiO₂ nanofiber membrane in PBS (37 °C).

| Time | Absorbance | Concentration | Normalized cumulative |
|---|---|---|---|
| (hrs) | (5 w % PLGA) | (μg/mL) | drug release (%) |
| 0 | 0.000 | 0.0000 | 0.000 |
| 5 | 0.025 | 46 | 23 |
| 10 | 0.060 | 116 | 58 |
| 15 | 0.095 | 186 | 93 |
| 20 | 0.130 | 256 | 128 |
| 25 | 0.160 | 316 | 158 |
| 30 | 0.190 | 376 | 188 |
| 35 | 0.215 | 426 | 212 |
| 40 | 0.240 | 476 | 238 |
| 45 | 0.260 | 516 | 258 |
| 50 | 0.275 | 546 | 273 |

### Results

Electrospun membranes formed continuous fiber networks with nanoscale to microscale fiber diameters and retained DOX@SiO₂ within the fiber matrix. Microscopy confirmed particle incorporation within fibers and supported a composite morphology suitable for implantable membrane handling.

Spectroscopic analysis identified polymer-associated functional group signatures and supported presence of doxorubicin-associated features in drug-loaded membranes. Calibration-based ultraviolet-visible analysis provided quantitative tracking of doxorubicin release into aqueous media over time.

Drug release studies produced a time-dependent release profile that included an initial release phase followed by a sustained release phase. Membranes formed from higher polymer concentration solutions exhibited slower release relative to membranes formed from lower polymer concentration solutions, consistent with increased diffusion resistance in the denser polymer matrix.

Cell exposure studies showed uptake of doxorubicin by A549 cells in the presence of drug-loaded membranes and supported local delivery to malignant cells in vitro.

What is claimed is:

1. A flexible nanofiber membrane comprising: an electrospun nanofiber matrix comprising a biodegradable polymer; and therapeutic-loaded nanoparticles retained within fibers of the electrospun nanofiber matrix, wherein the therapeutic-loaded nanoparticles comprise mesoporous silicon dioxide nanoparticles that retain a small-molecule therapeutic within internal pores, and wherein the flexible nanofiber membrane provides a dual-barrier release architecture in which release of the small-molecule therapeutic is governed by diffusion from the internal pores and by diffusion through, or degradation of, the electrospun nanofiber matrix.

2. The flexible nanofiber membrane of claim 1, wherein the biodegradable polymer comprises poly(lactic-co-glycolic acid) (PLGA).

3. The flexible nanofiber membrane of claim 1, wherein the small-molecule therapeutic comprises doxorubicin or a pharmaceutically acceptable salt thereof.

4. The flexible nanofiber membrane of claim 1, wherein the therapeutic-loaded nanoparticles are present in a mass fraction of 0.1% to 30% (w/w) relative to polymer of the electrospun nanofiber matrix.

5. The flexible nanofiber membrane of claim 1, wherein the electrospun nanofiber matrix comprises fibers having diameters from 50 nm to 10 μm.

6. The flexible nanofiber membrane of claim 1, wherein the electrospun nanofiber matrix comprises multiaxial fibers formed by coaxial electrospinning or triaxial electrospinning.

7. The flexible nanofiber membrane of claim 6, wherein the multiaxial fibers comprise a core region and an outer region that retain different payloads to provide staged time-release profiles.

8. The flexible nanofiber membrane of claim 7, wherein a second time-release profile begins at a delayed start point relative to a first time-release profile.

9. A method of local therapy comprising: placing the flexible nanofiber membrane of claim 1 at a target tissue site comprising a tumor bed or a surgical resection margin; and maintaining the flexible nanofiber membrane in contact with the target tissue site for a duration sufficient to locally deliver a therapeutic dose of the small-molecule therapeutic from the flexible nanofiber membrane.

10. The method of claim 9, wherein the target tissue site comprises a lung resection margin associated with non-small cell lung cancer.

11. The method of claim 9, wherein the flexible nanofiber membrane provides an initial release phase followed by a sustained release phase that maintains therapeutic exposure over multiple days.

12. The method of claim 9, wherein the flexible nanofiber membrane comprises a pH-responsive component that changes permeability or degradation rate in response to acidic pH lower than physiological pH at the target tissue site.

13. The method of claim 12, wherein the pH-responsive component is disposed in a layer that surrounds a compartment containing a payload, and wherein the layer increases porosity, permeability, or degradation rate under the acidic pH lower than physiological pH to initiate release of the payload at a delayed start point after placing the flexible nanofiber membrane at the target tissue site.

14. The method of claim 9, wherein the flexible nanofiber membrane comprises a targeting moiety disposed at an exterior surface of the flexible nanofiber membrane, and wherein the targeting moiety comprises an antibody, an antibody fragment, a peptide, an aptamer, or a small molecule ligand that binds a receptor expressed by malignant cells.

15. A method of making a flexible nanofiber membrane comprising: synthesizing mesoporous silicon dioxide nanoparticles using a surfactant-templated sol-gel process; loading a small-molecule therapeutic into pores of the mesoporous silicon dioxide nanoparticles to form therapeutic-loaded nanoparticles; preparing an electrospinning solution comprising a biodegradable polymer and the therapeutic-loaded nanoparticles; and electrospinning the electrospinning solution to form an electrospun nanofiber matrix that retains the therapeutic-loaded nanoparticles within fibers of the electrospun nanofiber matrix.

16. The method of claim 15, wherein synthesizing the mesoporous silicon dioxide nanoparticles comprises condensing silica from tetraethyl orthosilicate around cetyltrimethylammonium bromide micelles and removing the micelles to form pores.

17. The method of claim 15, wherein preparing the electrospinning solution comprises dissolving poly(lactic-co-glycolic acid) (PLGA) in a solvent system comprising dichloromethane and N,N-dimethylformamide at an 80:20 volume ratio and dispersing the therapeutic-loaded nanoparticles by sonication.

18. The method of claim 15, wherein electrospinning is performed using a flow rate of about 0.5 mL/hour, an applied voltage of about 13 kV, and a needle-to-collector distance of about 15 cm.

19. The method of claim 15, wherein electrospinning is performed using a multiaxial spinneret to form coaxial fibers or triaxial fibers.

20. The flexible nanofiber membrane of claim 1, wherein the electrospun nanofiber matrix comprises a polymer layer comprising poly(ethyleneimine), and wherein the polymer layer retains a nucleic acid payload comprising siRNA.
